(19) United States
(12) Patent Application Publication
(10) Pub. No.: US 2020/0376156 A1
(43) Pub. Date: Dec. 3, 2020

(54) METHODS OF ACCELERATING WOUND HEALING USING CANNABINOID COMPOSITIONS

(71) Applicant: Precision Biologics, Marietta, GA (US)

(72) Inventor: Peyton Palaio, Cumming, GA (US)

(21) Appl. No.: 16/879,638

(22) Filed: May 20, 2020

Related U.S. Application Data
(60) Provisional application No. 62/854,172, filed on May 29, 2019.

(51) Int. Cl. A61L 15/44; A61K 31/352; A61K 31/05

(52) U.S. Cl. CPC A61L 15/44; A61K 9/0014; A61K 31/05; A61K 31/352

(57) ABSTRACT

Provided are methods related to the treatment of epidermal wounds via topical application of a cannabinoid such as, for example, cannabidiol, cannabinol, tetrahydrocannabinol, and cannabigerol. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 62/854,172, filed on May 29, 2019, the contents of which are hereby incorporated by reference in their entirety.

### BACKGROUND

Wound healing is a complex process that occurs in almost all tissues after damage, aiming at repairing a lost or injured tissue. One of the determinative factors of wound healing is the rate at which the wound gains tensile strength against breakage. For example, with respect to skin wounds, if only the outer epidermis is damaged, keratinocytes migrate from the edge of the wound and eventually cover it, reforming the epidermis. In contrast, if multiple skin layers are injured, new connective tissues must first fill in the wound space. Such tissue, known as granulation tissue, is formed by deposition of extracellular matrix components, such as collagen, by fibroblasts that migrate into the wound space.

Although the human skin has a natural ability to promote self-regeneration after damage, this capacity can be compromised under specific conditions, for example, extensive skin loss, deep burns, chronic wounds, non-healing ulcers, and diabetes. An inappropriate healing process can lead the wound to enter into a chronic state, which increases the risk of infection and affects the patient's health and quality of life. Chronic wounds, such as venous ulcers and ischemic wounds, are characterized by the disruption of the normal regeneration process, usually as a result of bacterial colonization, vascular insufficiency, and diabetes, leading to a complicated and delayed healing process. Such wounds represent one of the most debilitating, painful, and costly skin conditions, being a critical medical and social problem for both patients and countries. Chronic wounds may also require longer hospitalization times and/or the employment of sophisticated and expensive wound care products (e.g., cellular tissue-engineered skin substitutes and medicated dressings), increasing medical costs. Although several clinical practices have been tested in order to prevent delayed healing and improve the healing process, the treatment options for chronic wounds are still very limited.

Practices and compounds arising from traditional medicine are an appealing strategy to target the acceleration of wound healing due to their therapeutic activities, availability, affordability, and relatively low cost. Such therapies involve the use of living organisms and natural compounds obtained from a wide variety of sources (e.g., animals, plants, fungi, and minerals). For example, aloe vera (AV), also known as Aloe barbadensis Miller, is an herb that is commonly used in skin healing. However, despite the tremendous potential of traditional therapies in wound care applications, improvements in the quality, efficacy, safety, and manufacturing processes are yet needed. Thus, there remains a need for compositions to accelerate healing of epidermal wounds and methods of making and using same. These needs and others are met by the present invention.

### SUMMARY

In accordance with the purpose(s) of the invention, as embodied and broadly described herein, the invention, in one aspect, relates to cannabinoid compositions and methods of treating epidermal wounds via topical application of a cannabinoid.

Thus, disclosed are compositions comprising a therapeutically effective amount of a first cannabinoid and an antimicrobial.

Also disclosed are compositions comprising CBD in an amount of from about 0.5 wt % to about 10 wt % and an antibiotic in an amount of from about 1 wt % to about 10 wt %, wherein the antibiotic consists essentially of bacitracin and polymixin B sulfate.

Also disclosed are compositions comprising CBG in an amount of from about 0.5 wt % to about 10 wt % and an antibiotic in an amount of from about 1 wt % to about 10 wt %, wherein the antibiotic consists essentially of bacitracin and polymixin B sulfate.

Also disclosed are methods for treating an epidermal wound on a subject, the method comprising topically administering to the epidermal wound a composition comprising a therapeutically effective amount of a first cannabinoid and an antimicrobial.

Also disclosed are methods for treating an epidermal wound on a subject, the method comprising topically administering to the epidermal wound a composition comprising CBD in an amount of from about 0.5 wt % to about 10 wt % and an antibiotic in an amount of from about 1 wt % to about 10 wt %, based upon the total weight of the composition administered, wherein the antibiotic consists essentially of bacitracin and polymixin B sulfate.

Also disclosed are methods for treating an epidermal wound on a subject, the method comprising topically administering to the epidermal wound a composition comprising CBG in an amount of from about 0.5 wt % to about 10 wt % and an antibiotic in an amount of from about 1 wt % to about 10 wt %, based upon the total weight of the composition administered, wherein the antibiotic consists essentially of bacitracin and polymixin B sulfate.

Still other objects and advantages of the present disclosure will become readily apparent by those skilled in the art from the following detailed description, wherein it is shown and described only the preferred aspects, simply by way of illustration of the best mode. As will be realized, the disclosure is capable of other and different aspects, and its several details are capable of modifications in various obvious respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present compositions, methods and systems may be understood more readily by reference to the following detailed description of preferred embodiments and the examples included therein and to the Figures and their previous and following description.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

Listed below are definitions of various terms used to describe this invention. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and the appended claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “substantially,” in, for example, the context “substantially free” refers to a composition having less than about 10% by weight, e.g., less than about 5%, less than about 1%, less than about 0.5% by weight, less than about 0.1% by weight, less than about 0.05% by weight, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a wound. The term “patient” includes human and veterinary subjects.

As used herein, the term “treatment” refers to the medical management of a patient with the intent to heal, ameliorate, stabilize, repair, or regenerate a wound, e.g., an epidermal wound. This term includes active treatment, that is, treatment directed specifically toward the improvement of a wound. In addition, this term includes palliative treatment, that is, treatment designed for the relief of symptoms rather than the elimination of the wound; and supportive treatment, that is, treatment employed to supplement another specific therapy directed toward the improvement of the wound. In various aspects, the term covers any treatment of a subject, including a mammal (e.g., a human), and includes: (i) healing, ameliorating, stabilizing, repairing, and/or regenerating the wound in a subject having the wound; or (ii) accelerating healing, ameliorating, repairing, and/or regenerating of the wound, i.e., increasing the speed at which the wound heals relative to wound healing in the absence of treatment. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a condition, e.g., a wound, or for prevention of worsening of a condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the injury; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of worsening of an injury.

As used herein, the term “individually effective amount” refers to an amount of a single component, e.g., a cannabinoid, in isolation, that is sufficient to achieve the desired result or to have an effect on an undesired condition. For example, an “individually therapeutically effective amount” refers to an amount of a single component that is sufficient to achieve the desired therapeutic result or to have an effect on undesired symptoms, but is generally insufficient to cause adverse side effects.

As used herein, the term “combinatorically effective amount” refers to an amount of multiple components, e.g., a cannabinoid and an antimicrobial, together, that is sufficient to achieve the desired result or to have an effect on an undesired condition. For example, a “combinatorically therapeutically effective amount” refers to an amount of multiple components in total that is sufficient to achieve the desired therapeutic result or to have an effect on undesired symptoms, but is generally insufficient to cause adverse side effects.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form, which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12th edition) , and they include, without limitation, medicaments; vitamins; mineral supplements; substances used for the treatment, prevention, diagnosis, cure or mitigation of a disease, illness, or injury; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease, illness, or injury; or substances which affect the structure or function of the body; or pro- drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “epidermal wound” refers to an injury to the skin that results in damage to the epidermis (the outermost layer of skin). The resulting damage need not be confined to the epidermis, however; rather, the damage may also be present in other layers of the skin such as, for example, the dermis (the inner layer of skin). Epidermal wounds are typically classified into one of four grades depending on the depth of the wound: (i) Grade I: wounds limited to the epithelium; (ii) Grade II: wounds extending into the dermis; (iii) Grade III: wounds extending into the subcutaneous tissue; and (iv) Grade IV (or full-thickness wounds): wounds wherein bones are exposed (e.g., a bony pressure point such as the greater trochanter or the sacrum). Examples of epidermal wounds include, but are not limited to, open wounds, closed wounds, scars, burns, ulcers, blisters, psoriasis wounds, diabetic wounds, surgical incision wounds, sores, post-surgical adhesions wounds, and wounds resulting from insect bites.

As used herein, the term “open wound” refers to an injury to the skin that is attributable to an object having caused the injury. Examples of open wounds include, but are not limited to, incisions, lacerations, abrasions, puncture wounds, penetration wounds, gunshot wounds and the like. Incisions or incised wounds may be caused by a clean, sharp-edged object such as, for example, a knife, a razor, or a glass splinter. Incisions involving only the epidermis can be classified as cuts. Lacerations are irregular wounds caused by a blunt impact to soft tissue that lies over hard tissue (e.g., laceration of the skin covering the skull) or tearing of skin and other tissues (e.g., tears caused by childbirth). Lacerations may show bridging, as connective tissue or blood vessels are flattened against the underlying hard surface. Abrasions (grazes) are superficial wounds in which the topmost layer of the skin (the epidermis) is scraped off, and are often caused by a sliding fall onto a rough surface. Puncture wounds may be caused by an object puncturing the skin, such as a nail or needle. Penetration wounds may be caused by an object such as a knife entering the body. Gunshot wounds are caused by a bullet or similar projectile driving into or through the body. As such, there may be two wounds, one at the site of entry and one at the site of exit, which is generally known as a through-and-through.

As used herein, the term “closed wound” refers to an injury to the skin that is attributable to a blunt force trauma that damages the underlying tissues. Examples of closed wounds include, but are not limited to, bruises, hematomas or blood tumors, injuries resulting from crushing or application of force.

As used herein, the term “healing” refers to a process to repair a wound (e.g., an epidermal wound) or to repair the resultant damage (e.g., damage to the skin).

As used herein, the phrase “inducing wound healing” or “accelerating wound healing” refers to either the induction of the formation of granulation tissue of wound contraction and/or the induction of re-epithelialization (i.e., the generation of new cells in the epithelium). Typically, wound healing is measured by decreasing wound area.

As used herein, the term “TRPV1 antagonist” refers to an agent that inhibits or decreases TRPV1 receptor activity. A TRPV1 antagonist includes both competitive and non-competitive agonists. Examples of TRPV1 antagonists include, but are not limited to, thiourea analogs (e.g., capsazepine, JYL-1421), urea analogs (e.g., A-425619, BCTC, JNJ-17203212, SB-705498), cinnamide analogs (e.g., SB-366791, AMG-9810), ruthenium red, quinazoline analogs (e.g., MK-2295), and benzimidazole analogs (e.g., AMG-2674).

Disclosed are also components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

### B. COMPOSITIONS

In one aspect, disclosed are compositions comprising a therapeutically effective amount of a first cannabinoid and an antimicrobial.

In one aspect, disclosed are compositions comprising CBD in an amount of from about 0.5 wt % to about 10 wt % and an antibiotic in an amount of from about 1 wt % to about 10 wt %, wherein the antibiotic consists essentially of bacitracin and polymixin B sulfate. In a further aspect, the composition comprises CBD in an amount of from about 0.5 wt % to about 8 wt % and an antibiotic in an amount of from about 1 wt % to about 8 wt %. In a still further aspect, the composition comprises CBD in an amount of from about 0.5 wt % to about 6 wt % and an antibiotic in an amount of from about 1 wt % to about 6 wt %. In yet a further aspect, the composition comprises CBD in an amount of from about 0.5 wt % to about 4 wt % and an antibiotic in an amount of from about 1 wt % to about 4 wt %. In an even further aspect, the composition comprises CBD in an amount of from about 0.5 wt % to about 2 wt % and an antibiotic in an amount of from about 1 wt % to about 2 wt %. In a still further aspect, the composition comprises CBD in an amount of from about 0.5 wt % to about 1 wt % and an antibiotic in an amount of from about 1 wt % to about 1 wt %. In yet a further aspect, the composition comprises CBD in an amount of from about 0.5 wt % to about 8 wt % and an antibiotic in an amount of from about 2 wt % to about 10 wt %. In an even further aspect, the composition comprises CBD in an amount of from about 0.5 wt % to about 6 wt % and an antibiotic in an amount of from about 4 wt % to about 10 wt %. In a still further aspect, the composition comprises CBD in an amount of from about 0.5 wt % to about 4 wt % and an antibiotic in an amount of from about 6 wt % to about 10 wt %. In yet a further aspect, the composition comprises CBD in an amount of from about 0.5 wt % to about 2 wt % and an antibiotic in an amount of from about 8 wt % to about 10 wt %.

In one aspect, disclosed are compositions comprising CBG in an amount of from about 0.5 wt % to about 10 wt % and an antibiotic in an amount of from about 1 wt % to about 10 wt %, wherein the antibiotic consists essentially of bacitracin and polymixin B sulfate. In a further aspect, the composition comprises CBG in an amount of from about 0.5 wt % to about 8 wt % and an antibiotic in an amount of from about 1 wt % to about 8 wt %. In a still further aspect, the composition comprises CBG in an amount of from about 0.5 wt % to about 6 wt % and an antibiotic in an amount of from about 1 wt % to about 6 wt %. In yet a further aspect, the composition comprises CBG in an amount of from about 0.5 wt % to about 4 wt % and an antibiotic in an amount of from about 1 wt % to about 4 wt %. In an even further aspect, the composition comprises CBG in an amount of from about 0.5 wt % to about 2 wt % and an antibiotic in an amount of from about 1 wt % to about 2 wt %. In a still further aspect, the composition comprises CBG in an amount of from about 0.5 wt % to about 1 wt % and an antibiotic in an amount of from about 1 wt % to about 1 wt %. In yet a further aspect, the composition comprises CBG in an amount of from about 0.5 wt % to about 8 wt % and an antibiotic in an amount of from about 2 wt % to about 10 wt %. In an even further aspect, the composition comprises CBG in an amount of from about 0.5 wt % to about 6 wt % and an antibiotic in an amount of from about 4 wt % to about 10 wt %. In a still further aspect, the composition comprises CBG in an amount of from about 0.5 wt % to about 4 wt % and an antibiotic in an amount of from about 6 wt % to about 10 wt %. In yet a further aspect, the composition comprises CBG in an amount of from about 0.5 wt % to about 2 wt % and an antibiotic in an amount of from about 8 wt % to about 10 wt %.

In various aspects, the first cannabinoid and the antimicrobial, together, are present in a therapeutically effective amount (i.e., the first cannabinoid and the antimicrobial are present in a combinatorically therapeutically effective amount). In a further aspect, the first cannabinoid is present in a therapeutically effective amount (i.e., the first cannabinoid is present in an individually therapeutically effective amount).

In various aspects, the first cannabinoid and the antimicrobial present at a ratio of from about 1:7 to about 7:1. In a further aspect, the first cannabinoid and the antimicrobial present at a ratio of from about 1:7 to about 5:1. In a still further aspect, the first cannabinoid and the antimicrobial present at a ratio of from about 1:7 to about 3:1. In yet a further aspect, the first cannabinoid and the antimicrobial present at a ratio of from about 1:7 to about 1:1.

In an even further aspect, the first cannabinoid and the antimicrobial present at a ratio of from about 1:5 to about 7:1. In a still further aspect, the first cannabinoid and the antimicrobial present at a ratio of from about 1:3 to about 7:1. In yet a further aspect, the first cannabinoid and the antimicrobial present at a ratio of from about 1:1 to about 7:1. In an even further aspect, the first cannabinoid and the antimicrobial present at a ratio of from about 1:5 to about 5:1. In a still further aspect, the first cannabinoid and the antimicrobial present at a ratio of from about 1:3 to about 3:1.

In various aspects, the composition contains a TRPV1 antagonist. In various further aspects, the composition does not contain a TRPV1 antagonist.

In various aspects, the composition is topical.

In various aspects, the composition further comprises a pharmaceutically active carrier. The pharmaceutically acceptable carrier can, for example, be any known carrier suitable for topical applications. Thus, in various further aspects, the pharmaceutically acceptable carrier is a pharmaceutically acceptable topical carrier.

In various aspects, compositions of the present invention can be in any form suitable for topical use such as, for example, an aerosol, cream, ointment, lotion, dusting powder, gel, jelly, and the like. These formulations can be prepared via conventional processing methods known to one skilled in the art.

In a further aspect, the composition is an ointment, a gel, a jelly, an oil, a cream, a paste, an aerosol foam, an aerosol spray, a lotion, or a powder. In a still further aspect, the composition is a cream.

In a further aspect, the composition further comprises an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others.

It is understood that the disclosed compositions can be employed in the disclosed methods of using.

### 1. Cannabinoids

In one aspect, the disclosed compositions comprise a therapeutically effective amount of a first cannabinoid. In a further aspect, the therapeutically effective amount is an individually therapeutically effective amount. In a still further aspect, the therapeutically effective amount is a combinatorically therapeutically effective amount.

In one aspect, the disclosed compositions comprise CBD in an amount of from about 0.5 wt % to about 10 wt %. In a further aspect, the disclosed compositions comprise CBD in an amount of from about 0.5 wt % to about 8 wt %. In a still further aspect, the disclosed compositions comprise CBD in an amount of from about 0.5 wt % to about 6 wt %. In yet a further aspect, the disclosed compositions comprise CBD in an amount of from about 0.5 wt % to about 4 wt %. In an even further aspect, the disclosed compositions comprise CBD in an amount of from about 0.5 wt % to about 2 wt %. In a still further aspect, the disclosed compositions comprise CBD in an amount of from about 0.5 wt % to about 1 wt %. In yet a further aspect, the disclosed compositions comprise CBD in an amount of from about 1 wt % to about 10 wt %. In an even further aspect, the disclosed compositions comprise CBD in an amount of from about 2 wt % to about 10 wt %. In a still further aspect, the disclosed compositions comprise CBD in an amount of from about 4 wt % to about 10 wt %. In yet a further aspect, the disclosed compositions comprise CBD in an amount of from about 6 wt % to about 10 wt %. In an even further aspect, the disclosed compositions comprise CBD in an amount of from about 8 wt % to about 10 wt %.

In one aspect, the disclosed compositions comprise CBG in an amount of from about 0.5 wt % to about 10 wt %. In a further aspect, the disclosed compositions comprise CBG in an amount of from about 0.5 wt % to about 8 wt %. In a still further aspect, the disclosed compositions comprise CBG in an amount of from about 0.5 wt % to about 6 wt %. In yet a further aspect, the disclosed compositions comprise CBG in an amount of from about 0.5 wt % to about 4 wt %. In an even further aspect, the disclosed compositions comprise CBG in an amount of from about 0.5 wt % to about 2 wt %. In a still further aspect, the disclosed compositions comprise CBG in an amount of from about 0.5 wt % to about 1 wt %. In yet a further aspect, the disclosed compositions comprise CBG in an amount of from about 1 wt % to about 10 wt %. In an even further aspect, the disclosed compositions comprise CBG in an amount of from about 2 wt % to about 10 wt %. In a still further aspect, the disclosed compositions comprise CBG in an amount of from about 4 wt % to about 10 wt %. In yet a further aspect, the disclosed compositions comprise CBG in an amount of from about 6 wt % to about 10 wt %. In an even further aspect, the disclosed compositions comprise CBG in an amount of from about 8 wt % to about 10 wt %.

Cannabis is a genus of flowering plants from order Rosales, family Cannabaceae, which includes three different species, Cannabis sativa, Cannabis indica and Cannabis ruderalis, which are indigenous to Central and South Asia. Cannabis has long been used for hemp fiber, for seed and seed oils, for medicinal purposes, and well as being a recreational drug. Pharmacologically, Cannabis contains 483 known chemical compounds, including at least 85 different cannabinoids. Cannabinoids, terpenoids, and other compounds are secreted by glandular trichomes that occur most abundantly on the floral calyxes and bracts of female plants.

Cannabinoids are a class of diverse chemical compounds that act on cannabinoid receptors on cells that repress neurotransmitter release in the brain. Cannabinoid receptors are of a class of cell membrane receptors under the G protein-coupled receptor superfamily. As is typical of G protein-coupled receptors, the cannabinoid receptors contain seven transmembrane spanning domains. There are currently two known subtypes of cannabinoid receptors, termed CB1 and CB2, with mounting evidence of more. The CB1 receptor is expressed mainly in the brain (central nervous system), but also in the lungs, liver and kidneys. The CB2 receptor is expressed mainly in the immune system and in hematopoietic cells.

The classical cannabinoids are derived from their respective 2-carboxylic acids (2-COOH) by decarboxylation, catalyzed by heat, light, or alkaline conditions. Phyto-cannabinoids (those derived from the Cannabis plant) include but not limited to: tetrahydrocannabinol (THC), tetrahydrocannabinolic acid (THCA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV) and cannabigerol monomethyl ether (CBGM).

The most notable cannabinoid is the phytocannabinoid A9-tetrahydrocannabinol (THC), which is the primary psychoactive component of the cannabis plant. THC has approximately equal affinity for the CB1 and CB2 receptors, and it possess activities as a psychoactive agent, analgesic, muscle relaxant, antispasmodic, bronchodilator, neuroprotective, antioxidant and antipruritic agent. Dronabinol is the International Nonproprietary Name (INN) for a pure isomer of THC, (−)-trans-A9- tetrahydrocannabinol. Synthesized dronabinol is marketed as MARINOL (a registered trademark of Solvay Pharmaceuticals). In the United States, MARINOL® is a Schedule III drug, available by prescription, considered to be non-narcotic and to have a low risk of physical or mental dependence. MARINOL® has been approved by the U.S. Food and Drug Administration (FDA) in the treatment of anorexia in AIDS patients, as well as for refractory nausea and vomiting of patients undergoing chemotherapy. An analog of dronabinol, Nabilone (a Schedule II drug), with therapeutic use as an antiemetic and as an adjunct analgesic for neuropathic pain, is available commercially in Canada under the trade name CESAMET®. CESAMET® has also received FDA approval and began marketing in the U.S. in 2006.

Cannabidiol (CBD) is another major phyto-cannabinoid, accounting for up to 40% of the plant's extract in selected cultivars. CBD is considered to have a wider scope of medical applications than THC. An orally- administered liquid containing CBD has received orphan drug status in the US, for use as a treatment for Dravet syndrome, under the trade name EPIDIOLEX®. Anandamide (N-arachidonoylethanolamine, AEA), one of the major components of endocannabinoid system, is a THC mimetic. Its effects can be either central, in the brain, or peripheral, in other parts of the body and are mediated primarily by CB 1 in the central nervous system, and CB2 in the periphery. However, short half-life due to the action of the enzyme fatty acid amide hydrolase (FAAH), presents a disadvantage for potential therapeutic use.

In a further aspect, the first cannabinoid is selected from Δ9-tetrahydrocannabinol (THC), cannabidiol (CBD), cannabinol (CBN), and cannabigerol (CBG), or a mixture thereof. In a still further aspect, the first cannabinoid is CBD. In yet a further aspect, the first cannabinoid is THC. In an even further aspect, the first cannabinoid is CBG. In a still further aspect, the first cannabinoid is CBN. In yet a further aspect, the first cannabinoid is a mixture of two or more of THC, CBD, CBN, and CBG. In an even further aspect, the first cannabinoid is a mixture of THC and CBD. In a still further aspect, the first cannabinoid is a mixture of THC and CBG. In yet a further aspect, the first cannabinoid is a mixture of THC and CBD. In an even further aspect, the first cannabinoid is a mixture of CBD and CBN. In a still further aspect, the first cannabinoid is a mixture of CBD and CBG. In yet a further aspect, the first cannabinoid is a mixture of CBN and CBG. In an even further aspect, the first cannabinoid is not a mixture of more than one cannabinoid.

In a further aspect, the first cannabinoid is present in an amount of from about 0.5 wt % to about 10 wt %. In a still further aspect, the first cannabinoid is present in an amount of from about 0.5 wt % to about 8 wt %. In yet a further aspect, the first cannabinoid is present in an amount of from about 0.5 wt % to about 6 wt %. In an even further aspect, the first cannabinoid is present in an amount of from about 0.5 wt % to about 4 wt %. In a still further aspect, the first cannabinoid is present in an amount of from about 0.5 wt % to about 2 wt %. In yet a further aspect, the first cannabinoid is present in an amount of from about 0.5 wt % to about 1 wt %. In an even further aspect, the first cannabinoid is present in an amount of from about 1 wt % to about 10 wt %. In a still further aspect, the first cannabinoid is present in an amount of from about 2 wt % to about 10 wt %. In yet a further aspect, the first cannabinoid is present in an amount of from about 4 wt % to about 10 wt %. In an even further aspect, the first cannabinoid is present in an amount of from about 6 wt % to about 10 wt %. In a still further aspect, the first cannabinoid is present in an amount of from about 8 wt % to about 10 wt %.

In various aspects, the composition further comprises a second cannabinoid, wherein the first cannabinoid and the second cannabinoid are different. In a further aspect, the second cannabinoid is selected from Δ9-tetrahydrocannabinol (THC), cannabinol (CBN), and cannabigerol (CBG), or a mixture thereof. In a still further aspect, the second cannabinoid is CBG. In yet a further aspect, the second cannabinoid is THC. In an even further aspect, the second cannabinoid is CBN. In a still further aspect, the second cannabinoid is a mixture of two or more of THC, CBN, and CBG. In yet a further aspect, the second cannabinoid is a mixture of THC and CBN. In an even further aspect, the second cannabinoid is a mixture of THC and CBG. In a still further aspect, the second cannabinoid is a mixture of CBN and CBG. In yet a further aspect, the second cannabinoid is not a mixture of more than one cannabinoid.

In various aspects, the first cannabinoid and the second cannabinoid, together, are present in a therapeutically effective amount (i.e., the first cannabinoid and the second cannabinoid are present in a combinatorically therapeutically effective amount). In a further aspect, the second cannabinoid is present in a therapeutically effective amount (i.e., the second cannabinoid is present in an individually therapeutically effective amount).

### 2. Antimicrobials

In one aspect, the disclosed compositions comprise an antibimicrobial. Examples of antimicrobials include, but are not limited to, germicides, antibiotics, antibacterials, antivirals and antifungals.

In one aspect, the disclosed compositions comprise an antibiotic in an amount of from about 1 wt % to about 10 wt %, wherein the antibiotic consists essentially of bacitracin and polymixin B sulfate. In a further aspect, the disclosed compositions comprise an antibiotic in an amount of from about 1 wt % to about 8 wt %. In a still further aspect, the disclosed compositions comprise an antibiotic in an amount of from about 1 wt % to about 6 wt %. In yet a further aspect, the disclosed compositions comprise an antibiotic in an amount of from about 1 wt % to about 4 wt %. In an even further aspect, the disclosed compositions comprise an antibiotic in an amount of from about 1 wt % to about 2 wt %. In a still further aspect, the disclosed compositions comprise an antibiotic in an amount of from about 2 wt % to about 10 wt %. In yet a further aspect, the disclosed compositions comprise an antibiotic in an amount of from about 4 wt % to about 10 wt %. In an even further aspect, the disclosed compositions comprise an antibiotic in an amount of from about 6 wt % to about 10 wt %. In a still further aspect, the disclosed compositions comprise an antibiotic in an amount of from about 8 wt % to about 10 wt %.

In a further aspect, the antimicrobial is an antibacterial. Examples of antibacterials include, but are not limited to, penicillins, cephalosporins, tetracyclines, quinolones, and aminoglycosides.

In a further aspect, the antimicrobial is an antiviral. Examples of antivirals include, but are not limited to, famciclovir, penciclovir, and acyclovir.

In a further aspect, the antimicrobial is an antifungal. Examples of antifungals include, but are not limited to, terbinafine, itraconazole, ketoconazole, fluconazole, oxiconazole, sulconazole, clotrimazole, miconazole, econazole, azanidazole, bifonazole, butoconazole, chlormidazole, fenticonazole, imazalil, isoconazole, neticonazole, sertaconazole, tioconazole, naftifine, griseofulvin, amorolfine, and sodium pyrithione, and derivatives thereof. In a still further aspect, the antifungal agent is selected from clotrimazole, econazole, micronazole, terbinafine, fluconazole, ketoconazole, and amphotericin.

In a further aspect, the antimicrobial is a germicide. Examples of germicides include, but are not limited to, benzimidazole-based germicides such as benomyl, carbendazim, fuberidazole, thiabendazole or thiophanate methyl; dicarboxyimide-based germicides such as chlozolinate, iprodione, procymidone or vinclozolin; DMI germicides such as imzalil, oxpoconazole, pefurazoate, prochloraz, triflumizole, triforine, pyrifenox, fenarimol, nuarimol, azaconazole, bitertanol, bromconazole, cyproconazole, difenoconazole, diniconazole, epoxyconazole, fenbuconazole, fluquinconazole, flusilazole, flutriafol, hexaconazole, imibenconazole, ipuconazole, metconazole, myclobutanil, penconazole, propiconazole, prothioconazole, simeconazole, tebuconazole, tetraconazole, triadimefon, triadimenol, triticonazole, etaconazole, furconazole-cis, ipconazole or imibenconazole; phenylamide-based germicides such as benalaxyl, furalaxyl, metalaxyl, metalaxyl-M, oxadixyl or ofurace, amine-based germicides such as aldimorph, dodemorph, fenpropimorph, tridemorph, fenpropidine, piperalin or spiroxamine; phosphorothiolate-based germicides such as EDDP, iprobenfos or pyrazophos; dithiolane-based germicides such as isoprothiolane; carboxamide-based germicides such as benodanil, boscalid, carboxin, fenfuran, flutolanil, furametpyr, mepronil, oxycarboxin, penthiopyrad or thifluzamide; hydroxy(2-amino)pyrimidines such as bupirimate, dimethirimol or ethirimol; AP germicides (anilinopyrimidines) such as cyprodinil, mepanipyrim or pyrimethanil; N-phenylcarbamates such as diethofencarb; and, QoI germicides (Qo inhibitors) such as azoxystrobin, picoxystrobin, pyraclostrobin, kresoxim-methyl, trfloxystrobin, dimoxystrobin, metominostrobin, orysastrobin, famoxadone, fluoxastrobin, fenamidone or metominofen; PP germicides (phenylpyrroles) such as fenpiconil or fludioxonil; quinoline-based germicides such as quinoxyfen; AH germicides (aromatic hydrocarbons) such as biphenyl, chloroneb, dichloran, quintozene, tecnazene or tolclofos-methyl; MRI-R germicides such as fthalide, pyroquilon or tricyclazole; MBI-D germicides such as carpropamid, diclocymet or fenoxanil; SBI agents such as fenhexamid, pyributicarb or terbinafine; phenylureas such as pencycuron; Qil germicides (Qi inhibitors) such as cyazofamid; benzamides such as zoxamide; enopyranurones such as blasticidin or mildiomycin; hexopyranosyls such as kasugamycin; giucopyranosyls such as streptomycin or validamycin; cyanoacetoamides such as cymoxanil; carbamates such as propamocarb, prothiocarb or polycarbamate; uncoupling agents such as binapacryl, dinocap, ferimzone or fluazinam; organic tin compounds such as triphenyltin acetate, triphenyltin chloride or triphenyltin hydroxide; phosphate esters such as phosphonic acid, tolclofos-methyl or fosetyl; phthalamides such as tecloftalam; benzotriazines such as triazoxide; benzene sulfonamides such as flusulfamide; pyridazinones such as diclomezine; CAA germicides (carbonic acid amides) such as dimethomorph, flumorph, benthiavalicarb, iprovalicarb or mandipropamide; tetracyclines such as oxytetracycline; thiocarbamates such as methasulfocarb; and, other compounds such as etridiazole, polyoxins, oxolinic acid, hydroxyisoxazole, octinoline, silthiofam, diflumetorim, acibenzolar-s-methyl, probenazole, tiadinil, ethaboxam, cyflufenamid, proquinazid, metrafenone, fluopicolide, cupric hydroxide, organic copper, sulfur, ferbam, manzeb, maneb, metiram, propineb, thiuram, zineb, ziram, captan, captafol, folpet, chlorothalonil, dichlofluanid, tolylfluanid, dodine, guazatine, iminoctadine acetate, iminoctadine dodecylbenzene sulfonate, anilazine, dithianon, chloropicrin, dazomet, metam sodium salt, chinomethionat, cyprofuram, silthiofam, and fluoroimide

In a further aspect, the antimicrobial is an antibiotic. Examples of antibiotics include, but are not limited to, gram-positive acting, bacteriocida antibiotics, lipopeptidal antibiotics, cyclic peptidal antibiotics, daptomycin and analogs thereof; anti-migraine agents; BIBM-4096BS, calcitonin gene-related proteins antagonists, sumatriptan succinate; antivirals, acyclovir, valacyclovir; atrial naturetic factor; argatroban; bisphosphonates, alendronate, clodronate, etidronate, ibandronate, incadronate, minodronate, neridronate, olpadronate, pamidronate, risedronate, tiludronate, zoledronate, EB1053, AND YH529; BIBN4096BS-(1-piperidinecarboxamide. n-[2-[[5-amino-1-[[4-(4-pyridinyl)-1-piperazinyl)carbonyl]pentyl]amino]1-[(3,5-dibromo-4-hydroxyphenyl)methyl]-2-oxoethyl]-4(1,4-dihydro-2-oxo-3(2H0-quinazolinyl)-. [R—(R*,S*)]—); calcitonin, salmon calcitonin, eel calcitonin, porcine calcitonin, human calcitonin; cholecystokinin (CCK) and CCK agonists, CCK-8; cromolyn sodium (sodium or disodium chromoglycate); CPHPC; cyclosporine; desferrioxamine (DFO); erythropoietin; exedin and exedin agonists, exendin-3, exendin-4; filgrastim; follicle stimulating hormone (recombinant and natural); gallium nitrate; glucagon; glucagon-like peptide 1 (GLP-1), glucagon, glucagon-like peptide 2 (GLP-2); glucocerebrosidase; gonadotropin releasing hormone; growth hormone releasing factor; growth hormone releasing hormones; growth hormone, human growth hormone (hGH), recombinant human growth hormone (rhGH), bovine growth hormone, porcine growth hormone; heparin, unfractionated heparin, heparinoids, dermatans, chondroitins, low molecular weight heparin, very low molecular weight heparin, ultra low molecular weight heparin, synthetic heparin, fondiparinux; insulin, porcine insulin, bovine insulin, human insulin, human recombinant insulin, optionally having counter ions including zinc, sodium, calcium and ammonium; insulin-like growth factor, IGF-1; interferons, a-interferon, β-interferon, omega interferon, y interferon; interleukin-1; interleukin-2; interleukin-11; interleukin-21; leutinizing hormone and leutinizing hormone releasing hormone; leptin (OB protein); methyphenidate salt; monoclonal antibodies, retuxin, tnf-alpha soluble receptors; oxytocin; parathyroid hormone (PTH), PTH 1-34 and PTH 1-38, and other fragments of parathyroid hormone; peptide YY (PYY), PYY agonists, PYY3-36; dipeptidyl peptidase IV (DPP-4) inhibitors; prostaglandins; protease inhibitors; somatostatin; thrombopoietin; vancomycin; vasopressin; vitamins; vaccines, anthrax vaccines, Y. pestis vaccines, influenza vaccines, herpes vaccines; analogs, fragments, mimetics and polyethylene glycol-modified derivatives of any of the above compounds, and any combination thereof. In a still further aspect, the antibiotic is a penicillin. In yet a further aspect, the antibiotic is selected from a cephalosporin, a macrolide, a fluoroquinolone, a sulfonamide, a tetracycline, and an aminoglycoside, or a combination thereof. In an even further aspect, the antibiotic is selected from bacitracin, neomycin sulfate, polymixin B sulfate, penicillin, amoxicillin, cephalexin, erythromycin, clarithromycin, azithromycin, ciprofloxacin, levofloxacin, ofloxacin, co-trimoxazole, trimethoprim, tetracyclin, doxycycline, gentamicin, and tobramycin, or a combination thereof

In a further aspect, the antibiotic consists essentially of bacitracin and polymixin B sulfate.

In a further aspect, the antibiotic consists essentially of bacitracin, polymixin B, and neomycin sulfate. In a still further aspect, the antibiotic consists essentially of bacitracin and neomycin sulfate. In yet a further aspect, the antibiotic consists essentially of polymixin B and neomycin sulfate.

In a further aspect, the antimicrobial is present in an amount of from about 1 wt % to about 10 wt %.

### 3. Optional Additives

In various aspects, the disclosed composition further comprises one or more additives. Thus, in one aspect, the disclosed composition further comprises one or more of an anti-infective agent, an anti-inflammatory agent, a neuropathic pain agent, an agent to increase or decrease pore size, and a steroid.

In a further aspect, the additive is present in an amount of from about 0.01 wt % to about 10 wt % of the composition. In a still further aspect, the additive is present in an amount of from about 0.01 wt % to about 8 wt % of the composition. In yet a further aspect, the additive is present in an amount of from about 0.01 wt % to about 6 wt % of the composition. In an even further aspect, the additive is present in an amount of from about 0.01 wt % to about 4 wt % of the composition. In a still further aspect, the additive is present in an amount of from about 0.01 wt % to about 2 wt % of the composition. In yet a further aspect, the additive is present in an amount of from about 0.01 wt % to about 1 wt % of the composition. In an even further aspect, the additive is present in an amount of from about 0.1 wt % to about 10 wt % of the composition. In a still further aspect, the additive is present in an amount of from about 1 wt % to about 10 wt % of the composition. In yet a further aspect, the additive is present in an amount of from about 2 wt % to about 10 wt % of the composition. In an even further aspect, the additive is present in an amount of from about 4 wt % to about 10 wt % of the composition. In a still further aspect, the additive is present in an amount of from about 6 wt % to about 10 wt % of the composition. In yet a further aspect, the additive is present in an amount of from about 8 wt % to about 10 wt % of the composition.

In a further aspect, the disclosed composition further comprises an anti-infective agent. Examples of anti-infective agents include, but are not limited to, amebicides (e.g., chloroquine phosphate, iodoquinol, metronidazole, paromomycin), aminoglycosides (e.g., neomycin, amikacin, gentamicin, kanamycin, streptomycin, tobramycin), anthelmintics (e.g., benzimidazoles, ivermectin, praziquantel, pyrantel), antifungal agents (e.g., terbinafine, anidulafungin, caspofungin, micafungin sodium, flucytosine, griseofulvin, ketoconazole, amphotericin B, nystatin, fluconazole, isavuconazonium sulfate, itraconazole, posaconazole, voriconazole), antiprotozoals (e.g., atovaquone, benznidazole, miltefosine, nitazoxanide, pentamidine isethionate, secnidazole, tinidazole), antiviral agents (e.g., acyclovir (acycloguanosine) (systemic), famciclovir, valacyclovir, cidofovir, entecavir, foscarnet sodium (phosphonoformic acid; PFA), ganciclovir (DHPG), hepatitis C virus direct-acting antivirals, letermovir, oseltamivir, peramivir, ribavirinrimantadine hydrochloride, telbivudine, valganciclovir, zanamivir, adefovir dipivoxil, amantadine hydrochloride), bacitracin, carbapenems (e.g., doripenem, ertapenem, imipenem-cilastatin, meropenem), cephalosporins (e.g., cefadroxil, cefazolin, cephalexin, cefprozil, cefuroxime, cefdinir, cefixime, cefotaxime, cefpodoxime, ceftazidime, ceftibuten, ceftriaxone, cefepime, ceftaroline), chloramphenicol, colistimethate sodium, fluoroquinolones (e.g., ciprofloxacin, gemifloxacin, levofloxacin, moxifloxacin, norfloxacin, ofloxacin), folate antagonists (e.g., trimethoprim), glycylcyclines (e.g., tigecycline), ketolides (e.g., telithromycin), leprostatics (e.g., dapsone), lincosamides (e.g., clindamycin, lincomycin), lipoglycopeptides (e.g., dalbavancin, oritavancin, telavancin), lipopeptides (e.g., daptomycin), macrolides (e.g., azithromycin, clarithromycin, erythromycin, fidaxomicin), methenamines, metronidazole, monobactams (e.g., aztreonam), nitrofurans (e.g., nitrofurantion), oxazolidinones (e.g., linezolid, tedizolid phosphate), penicillins (e.g., penicillin G, penicillin V, dicloxacillin, nafcillin, oxacillin, amoxicillin, amoxicillin/potassium clavulanate, ampicillin, ampicillin/sulbactam, piperacillin/tazobactam sodium, ticarcillin/potassium clavulanate), polymyxin B sulfate, rifaximin, streptogramins (e.g., quinupristin/dalfopristin), sulfadiazine, tetracyclines (e.g., demeclocycline, doxycycline, minocycline, tetracycline), and vancomycin.

In a further aspect, the disclosed composition further comprises an anti-inflammatory agent. Examples of anti-inflammatory agents include, but are not limited to, glucocorticoids (e.g., betamethasone, budesonide, cortisone, defalzacort, dexamethasone, hydrocortisone, methylprednisolone, prednisolone, prednisone, triamcinolone), NSAIDs (e.g., acetic acids such as diclofenax, indomethacin, sulindac, and tolmetin; COX-2 inhibitors such as celecoxib; fenamates such as meclofenamate and mefenamic acid; naphthylalkanones such as nabumetone; oxicams such as piroxicam and meloxicam; propionic acids such as fenoprofen, flurbiprofen, ibuprofen, ketoprofen, naproxen, and oxaprozin; pyranocarboxylic acids such as etodolac; pyrrolizine carboxylic acids such as ketorolac), and salicylates (e.g., aspirin, choline magnesium trisalicylate, diflunisal, magnesium salicylate, salsalate).

In a further aspect, the disclosed composition further comprises a neuropathic pain agent. Examples of neuropathic agents include, but are not limited to, tricyclic antidepressants (e.g., amitriptyline), anticonvulsants (e.g., gabapentin), local anesthetics (e.g., lidocaine), corticosteroids, and capsaicin cream.

In a further aspect, the disclosed composition further comprises an agent to increase or decrease pore size. Examples of agents that increase or decrease pore size include, but are not limited to, menthol and camphor.

In a further aspect, the disclosed composition further comprises a steroid. Examples of steroids include, but are not limited to, corticosteroids (e.g., gluticocorticoids such as hydrocortisone, cortisone, ethamehtasoneb, prednisone, prednisolone, triamcinolone, and dexamethasone, mineralocorticoids such as fludrocortisone, bethamethasone, and methylprednisolone), testosterone, cholic acid, dexamethasone, lanosterol, progesterone, medrogestone, β-sitosterol, cholesterol, glucocorticoids (e.g., alclometasone, prednisone, dexamethasone, triamcinolone), vitamin D (e.g., dihydrotachysterol), androgens (e.g., apopotone, oxandrolone, oxabolone, testosterone, nandrolone), oestrogens (e.g., diethylstilbestrol), and progestins (e.g., danazol, norethindrone, medroxyprogesterone acetate, and 17-hydroxyprogesterone caproate).

### C. ARTICLES COMPRISING THE COMPOSITIONS

In one aspect, disclosed are articles comprising a disclosed composition. Thus, in various aspects, disclosed are articles comprising a composition, wherein the composition comprises a therapeutically effective amount of a first cannabinoid and an antimicrobial. In a further aspect, disclosed are articles comprising a composition, wherein the composition comprises CBD in an amount of from about 0.5 wt % to about 10 wt % and an antibiotic in an amount of from about 1 wt % to about 10 wt %, wherein the antibiotic consists essentially of bacitracin and polymixin B sulfate. In a further aspect, disclosed are articles comprising a composition, wherein the composition comprises CBG in an amount of from about 0.5 wt % to about 10 wt % and an antibiotic in an amount of from about 1 wt % to about 10 wt %, wherein the antibiotic consists essentially of bacitracin and polymixin B sulfate.

In one aspect, disclosed are articles comprising a therapeutically effective amount of a first cannabinoid and an antimicrobial. In a further aspect, the first cannabinoid and the antimicrobial are present as a composition.

In one aspect, disclosed are articles comprising CBD in an amount of from about 0.5 wt % to about 10 wt % and an antibiotic in an amount of from about 1 wt % to about 10 wt %, wherein the antibiotic consists essentially of bacitracin and polymixin B sulfate. In a further aspect, CBD and the antimicrobial are present as a composition.

In one aspect, disclosed are articles comprising CBG in an amount of from about 0.5 wt % to about 10 wt % and an antibiotic in an amount of from about 1 wt % to about 10 wt %, wherein the antibiotic consists essentially of bacitracin and polymixin B sulfate. In a further aspect, CBG and the antimicrobial are present as a composition.

In a further aspect, the article is a wound care product. Examples of wound care products include, but are not limited to, debriding agents, polyurethane foams, hydrogels, transparent films, hydrocolloids, hydro-fibers, alginates, dressings, collagen, lidocaine, platelet-derived growth factors, bandages, and wound dressing materials. Thus, in various aspects, the first cannabinoid and the antimicrobial may be present on a wound care product. In a further aspect, the first cannabinoid and the antimicrobial may be present as a composition on a wound care product.

In a further aspect, the wound care product is a bandage or a wound dressing material. Thus, in various aspects, the first cannabinoid and the antimicrobial may be present on a bandage or a wound dressing material. In a further aspect, the first cannabinoid and the antimicrobial may be present as a composition on a bandage or a wound dressing material.

### D. METHODS OF USING THE COMPOSITIONS

The disclosed compositions of the invention are useful in treating or controlling epidermal wounds such as, for example, burns, sores, lacerations, blisters, insect bites, surgical incisions, and ulcers.

To treat or control the wound, the compositions are administered to a subject in need thereof, such as a mammal, e.g., a human. The subject can be a human, non-human primate, horse, pig, rabbit, dog, sheep, goat, cow, cat, guinea pig, or rodent. The term does not denote a particular age or sex. Thus, adult and newborn subjects, whether male or female, are intended to be covered. The subject is preferably a mammal, such as a human. Prior to administering the compositions, the subject can be diagnosed with a need for treatment of an epidermal wound, such as, for example, a burn, a sore, a laceration, a blister, an insect bite, a surgical incision, and an ulcer.

The compounds or compositions can be administered to the subject via topical administration. Administration can be continuous or intermittent. A preparation can be administered therapeutically; that is, administered to treat an existing disease or condition. The therapeutically effective amount or dosage of the composition can vary within wide limits. Such a dosage is adjusted to the individual requirements in each particular case including the specific composition(s) being administered and the condition being treated, as well as the patient being treated. In general, single dose compositions can contain such amounts or submultiples thereof of the composition to make up the daily dose. The dosage can be adjusted by the individual physician in the event of any contraindications. Dosage can vary, and can be administered in one or more dose administrations daily, for one or several days.

In a further aspect, the subject is a mammal. In a still further aspect, the mammal is a human.

In a further aspect, the epidermal wound is selected from a burn, a sore, a laceration, a blister, an insect bite, a surgical incision, and an ulcer.

### 1. Treatment Methods

The compounds and compositions disclosed herein are useful for treating, repairing, regenerating, or accelerating healing of epidermal wounds. Thus, provided is a method comprising administering a therapeutically effective amount of a first cannabinoid and an antimicrobial to a subject. In a further aspect, the method can be a method for treating an epidermal wound.

### a. Treating an Epidermal Wound

In one aspect, disclosed are methods for treating an epidermal wound on a subject, the method comprising topically administering to the epidermal wound an effective amount of a composition comprising a first cannabinoid and an antimicrobial. Examples of skin ailments include, but are not limited to, burns, sores, lacerations, blisters, insect bites, surgical incisions, and ulcers. In a further aspect, the skin ailment is an ulcer. In a still further aspect, the skin ailment is a diabetic ulcer.

In one aspect, disclosed are methods for treating an epidermal wound on a subject, the method comprising topically administering to the epidermal wound a composition comprising CBD in an amount of from about 0.5 wt % to about 10 wt % and an antibiotic in an amount of from about 1 wt % to about 10 wt %, based upon the total weight of the composition administered, wherein the antibiotic consists essentially of bacitracin and polymixin B sulfate. In a further aspect, the method comprises topically administering to the epidermal wound a composition comprising CBD in an amount of from about 0.5 wt % to about 8 wt% and an antibiotic in an amount of from about 1 wt % to about 8 wt %, based upon the total weight of the composition administered. In a still further aspect, the method comprises topically administering to the epidermal wound a composition comprising CBD in an amount of from about 0.5 wt % to about 6 wt % and an antibiotic in an amount of from about 1 wt % to about 6 wt %, based upon the total weight of the composition administered. In yet a further aspect, the method comprises topically administering to the epidermal wound a composition comprising CBD in an amount of from about 0.5 wt % to about 4 wt % and an antibiotic in an amount of from about 1 wt % to about 4 wt %, based upon the total weight of the composition administered. In an even further aspect, the method comprises topically administering to the epidermal wound a composition comprising CBD in an amount of from about 0.5 wt % to about 2 wt % and an antibiotic in an amount of from about 1 wt % to about 2 wt %, based upon the total weight of the composition administered. In a still further aspect, the method comprises topically administering to the epidermal wound a composition comprising CBD in an amount of from about 0.5 wt % to about 1 wt % and an antibiotic in an amount of from about 1 wt % to about 1 wt %, based upon the total weight of the composition administered. In yet a further aspect, the method comprises topically administering to the epidermal wound a composition comprising CBD in an amount of from about 0.5 wt % to about 8 wt % and an antibiotic in an amount of from about 2 wt % to about 10 wt %, based upon the total weight of the composition administered. In an even further aspect, the method comprises topically administering to the epidermal wound a composition comprising CBD in an amount of from about 0.5 wt % to about 6 wt % and an antibiotic in an amount of from about 4 wt % to about 10 wt %, based upon the total weight of the composition administered. In a still further aspect, the method comprises topically administering to the epidermal wound a composition comprising CBD in an amount of from about 0.5 wt % to about 4 wt % and an antibiotic in an amount of from about 6 wt % to about 10 wt %, based upon the total weight of the composition administered. In yet a further aspect, the method comprises topically administering to the epidermal wound a composition comprising CBD in an amount of from about 0.5 wt % to about 2 wt % and an antibiotic in an amount of from about 8 wt % to about 10 wt %, based upon the total weight of the composition administered.

In one aspect, disclosed are methods for treating an epidermal wound on a subject, the method comprising topically administering to the epidermal wound a composition comprising CBG in an amount of from about 0.5 wt % to about 10 wt % and an antibiotic in an amount of from about 1 wt % to about 10 wt %, based upon the total weight of the composition administered, wherein the antibiotic consists essentially of bacitracin and polymixin B sulfate. In a further aspect, the method comprises topically administering to the epidermal wound a composition comprising CBG in an amount of from about 0.5 wt % to about 8 wt % and an antibiotic in an amount of from about 1 wt % to about 8 wt %, based upon the total weight of the composition administered. In a still further aspect, the method comprises topically administering to the epidermal wound a composition comprising CBG in an amount of from about 0.5 wt % to about 6 wt % and an antibiotic in an amount of from about 1 wt % to about 6 wt %, based upon the total weight of the composition administered. In yet a further aspect, the method comprises topically administering to the epidermal wound a composition comprising CBG in an amount of from about 0.5 wt % to about 4 wt % and an antibiotic in an amount of from about 1 wt % to about 4 wt %, based upon the total weight of the composition administered. In an even further aspect, the method comprises topically administering to the epidermal wound a composition comprising CBG in an amount of from about 0.5 wt % to about 2 wt % and an antibiotic in an amount of from about 1 wt % to about 2 wt %, based upon the total weight of the composition administered. In a still further aspect, the method comprises topically administering to the epidermal wound CBG in an amount of from about 0.5 wt % to about 1 wt % and an antibiotic in an amount of from about 1 wt % to about 1 wt %, based upon the total weight of the composition administered. In yet a further aspect, the method comprises topically administering to the epidermal wound a composition comprising CBG in an amount of from about 0.5 wt % to about 8 wt % and an antibiotic in an amount of from about 2 wt % to about 10 wt %, based upon the total weight of the composition administered. In an even further aspect, the method comprises topically administering to the epidermal wound a composition comprising CBG in an amount of from about 0.5 wt % to about 6 wt % and an antibiotic in an amount of from about 4 wt % to about 10 wt %, based upon the total weight of the composition administered. In a still further aspect, the method comprises topically administering to the epidermal wound a composition comprising CBG in an amount of from about 0.5 wt % to about 4 wt % and an antibiotic in an amount of from about 6 wt % to about 10 wt %, based upon the total weight of the composition administered. In yet a further aspect, the method comprises topically administering to the epidermal wound a composition comprising CBG in an amount of from about 0.5 wt % to about 2 wt % and an antibiotic in an amount of from about 8 wt % to about 10 wt %, based upon the total weight of the composition administered.

In a further aspect, the epidermal wound is a bacterial infection. In a still further aspect, the epidermal wound is selected from a burn, a sore, a laceration, a blister, an insect bite, a surgical incision, and an ulcer.

In a further aspect, the subject is a mammal. In a still further aspect, the subject is a human.

In a further aspect, the first cannabinoid is selected from β9-tetrahydrocannabinol (THC), cannabidiol (CBD), cannabinol (CBN), and cannabigerol (CBG), or a mixture thereof. In a still further aspect, the first cannabinoid is CBD. In yet a further aspect, the first cannabinoid is THC. In an even further aspect, the first cannabinoid is CBG. In a still further aspect, the first cannabinoid is CBN. In yet a further aspect, the first cannabinoid is a mixture of two or more of THC, CBD, CBN, and CBG. In an even further aspect, the first cannabinoid is a mixture of THC and CBD. In a still further aspect, the first cannabinoid is a mixture of THC and CBG. In yet a further aspect, the first cannabinoid is a mixture of THC and CBD. In an even further aspect, the first cannabinoid is a mixture of CBD and CBN. In a still further aspect, the first cannabinoid is a mixture of CBD and CBG. In yet a further aspect, the first cannabinoid is a mixture of CBN and CBG. In an even further aspect, the first cannabinoid is not a mixture of more than one cannabinoid.

In a further aspect, the first cannabinoid is administered in an amount of from about 0.5 wt % to about 10 wt %. In a still further aspect, the first cannabinoid is administered in an amount of from about 0.5 wt % to about 8 wt %. In yet a further aspect, the first cannabinoid is administered in an amount of from about 0.5 wt % to about 6 wt %. In an even further aspect, the first cannabinoid is administered in an amount of from about 0.5 wt % to about 4 wt %. In a still further aspect, the first cannabinoid is administered in an amount of from about 0.5 wt % to about 2 wt %. In yet a further aspect, the first cannabinoid is administered in an amount of from about 0.5 wt % to about 1 wt %. In an even further aspect, the first cannabinoid is administered in an amount of from about 1 wt % to about 10 wt %. In a still further aspect, the first cannabinoid is administered in an amount of from about 2 wt % to about 10 wt %. In yet a further aspect, the first cannabinoid is administered in an amount of from about 4 wt % to about 10 wt %. In an even further aspect, the first cannabinoid is administered in an amount of from about 6 wt % to about 10 wt %. In a still further aspect, the first cannabinoid is administered in an amount of from about 8 wt % to about 10 wt %.

In a further aspect, the antimicrobial is an antibacterial. Examples of antibacterials include, but are not limited to, penicillins, cephalosporins, tetracyclines, quinolones, and aminoglycosides.

In a further aspect, the antimicrobial is an antiviral. Examples of antivirals include, but are not limited to, famciclovir, penciclovir, and acyclovir.

In a further aspect, the antimicrobial is an antifungal. Examples of antifungals include, but are not limited to, terbinafine, itraconazole, ketoconazole, fluconazole, oxiconazole, sulconazole, clotrimazole, miconazole, econazole, azanidazole, bifonazole, butoconazole, chlormidazole, fenticonazole, imazalil, isoconazole, neticonazole, sertaconazole, tioconazole, naftifine, griseofulvin, amorolfine, and sodium pyrithione, and derivatives thereof. In a still further aspect, the antifungal agent is selected from clotrimazole, econazole, micronazole, terbinafine, fluconazole, ketoconazole, and amphotericin.

In a further aspect, the antimicrobial is a germicide. Examples of germicides include, but are not limited to, benzimidazole-based germicides such as benomyl, carbendazim, fuberidazole, thiabendazole or thiophanate methyl; dicarboxyimide-based germicides such as chlozolinate, iprodione, procymidone or vinclozolin; DMI germicides such as imzalil, oxpoconazole, pefurazoate, prochloraz, triflumizole, triforine, pyrifenox, fenarimol, nuarimol, azaconazole, bitertanol, bromconazole, cyproconazole, difenoconazole, diniconazole, epoxyconazole, fenbuconazole, fluquinconazole, flusilazole, flutriafol, hexaconazole, imibenconazole, ipuconazole, metconazole, myclobutanil, penconazole, propiconazole, prothioconazole, simeconazole, tebuconazole, tetraconazole, triadimefon, triadimenol, triticonazole, etaconazole, furconazole-cis, ipconazole or imibenconazole; phenylamide-based germicides such as benalaxyl, furalaxyl, metalaxyl, metalaxyl-M, oxadixyl or ofurace, amine-based germicides such as aldimorph, dodemorph, fenpropimorph, tridemorph, fenpropidine, piperalin or spiroxamine; phosphorothiolate-based germicides such as EDDP, iprobenfos or pyrazophos; dithiolane-based germicides such as isoprothiolane; carboxamide-based germicides such as benodanil, boscalid, carboxin, fenfuran, flutolanil, furametpyr, mepronil, oxycarboxin, penthiopyrad or thifluzamide; hydroxy(2-amino)pyrimidines such as bupirimate, dimethirimol or ethirimol; AP germicides (anilinopyrimidines) such as cyprodinil, mepanipyrim or pyrimethanil; N-phenylcarbamates such as diethofencarb; and, Qol germicides (Qo inhibitors) such as azoxystrobin, picoxystrobin, pyraclostrobin, kresoxim-methyl, trfloxystrobin, dimoxystrobin, metominostrobin, orysastrobin, famoxadone, fluoxastrobin, fenamidone or metominofen; PP germicides (phenylpyrroles) such as fenpiconil or fludioxonil; quinoline-based germicides such as quinoxyfen; AH germicides (aromatic hydrocarbons) such as biphenyl, chloroneb, dichloran, quintozene, tecnazene or tolclofos-methyl; MRI-R germicides such as fthalide, pyroquilon or tricyclazole; MBI-D germicides such as carpropamid, diclocymet or fenoxanil; SBI agents such as fenhexamid, pyributicarb or terbinafine; phenylureas such as pencycuron; Qil germicides (Qi inhibitors) such as cyazofamid; benzamides such as zoxamide; enopyranurones such as blasticidin or mildiomycin; hexopyranosyls such as kasugamycin; giucopyranosyls such as streptomycin or validamycin; cyanoacetoamides such as cymoxanil; carbamates such as propamocarb, prothiocarb or polycarbamate; uncoupling agents such as binapacryl, dinocap, ferimzone or fluazinam; organic tin compounds such as triphenyltin acetate, triphenyltin chloride or triphenyltin hydroxide; phosphate esters such as phosphonic acid, tolclofos-methyl or fosetyl; phthalamides such as tecloftalam; benzotriazines such as triazoxide; benzene sulfonamides such as flusulfamide; pyridazinones such as diclomezine; CAA germicides (carbonic acid amides) such as dimethomorph, flumorph, benthiavalicarb, iprovalicarb or mandipropamide; tetracyclines such as oxytetracycline; thiocarbamates such as methasulfocarb; and, other compounds such as etridiazole, polyoxins, oxolinic acid, hydroxyisoxazole, octinoline, silthiofam, diflumetorim, acibenzolar-s-methyl, probenazole, tiadinil, ethaboxam, cyflufenamid, proquinazid, metrafenone, fluopicolide, cupric hydroxide, organic copper, sulfur, ferbam, manzeb, maneb, metiram, propineb, thiuram, zineb, ziram, captan, captafol, folpet, chlorothalonil, dichlofluanid, tolylfluanid, dodine, guazatine, iminoctadine acetate, iminoctadine dodecylbenzene sulfonate, anilazine, dithianon, chloropicrin, dazomet, metam sodium salt, chinomethionat, cyprofuram, silthiofam, and fluoroimide

In a further aspect, the antimicrobial is an antibiotic. Examples of antibiotics include, but are not limited to, gram-positive acting, bacteriocida antibiotics, lipopeptidal antibiotics, cyclic peptidal antibiotics, daptomycin and analogs thereof; anti-migraine agents; BIBM-4096BS, calcitonin gene-related proteins antagonists, sumatriptan succinate; antivirals, acyclovir, valacyclovir; atrial naturetic factor; argatroban; bisphosphonates, alendronate, clodronate, etidronate, ibandronate, incadronate, minodronate, neridronate, olpadronate, pamidronate, risedronate, tiludronate, zoledronate, EB1053, AND YH529; BIBN-4096BS-(1-piperidinecarboxamide. n-[2-[[5-amino-1-[[4-(4-pyridinyl)-1-piperazinyl) carbonyl]pentyl]amino]-1-[(3,5-dibromo-4-hydroxyphenyl) methyl]-2-oxoethyl]-4(1,4-dihydro-2-oxo-3(2H0-quinazolinyl)-. [R—(R*,S*)]—); calcitonin, salmon calcitonin, eel calcitonin, porcine calcitonin, human calcitonin; cholecystokinin (CCK) and CCK agonists, CCK-8; cromolyn sodium (sodium or disodium chromoglycate); CPHPC; cyclosporine; desferrioxamine (DFO); erythropoietin; exedin and exedin agonists, exendin-3, exendin-4; filgrastim; follicle stimulating hormone (recombinant and natural); gallium nitrate; glucagon; glucagon-like peptide 1 (GLP-1), glucagon, glucagon-like peptide 2 (GLP-2); glucocerebrosidase; gonadotropin releasing hormone; growth hormone releasing factor; growth hormone releasing hormones; growth hormone, human growth hormone (hGH), recombinant human growth hormone (rhGH), bovine growth hormone, porcine growth hormone; heparin, unfractionated heparin, heparinoids, dermatans, chondroitins, low molecular weight heparin, very low molecular weight heparin, ultra low molecular weight heparin, synthetic heparin, fondiparinux; insulin, porcine insulin, bovine insulin, human insulin, human recombinant insulin, optionally having counter ions including zinc, sodium, calcium and ammonium; insulin-like growth factor, IGF-1; interferons, a-interferon, (3-interferon, omega interferon, y interferon; interleukin-1; interleukin-2; interleukin-11; interleukin-21; leutinizing hormone and leutinizing hormone releasing hormone; leptin (OB protein); methyphenidate salt; monoclonal antibodies, retuxin, tnf-alpha soluble receptors; oxytocin; parathyroid hormone (PTH), PTH 1-34 and PTH 1-38, and other fragments of parathyroid hormone; peptide YY (PYY), PYY agonists, PYY3-36; dipeptidyl peptidase IV (DPP-4) inhibitors; prostaglandins; protease inhibitors; somatostatin; thrombopoietin; vancomycin; vasopressin; vitamins; vaccines, anthrax vaccines, Y. pestis vaccines, influenza vaccines, herpes vaccines; analogs, fragments, mimetics and polyethylene glycol-modified derivatives of any of the above compounds, and any combination thereof. In a still further aspect, the antibiotic is a penicillin. In yet a further aspect, the antibiotic is selected from a cephalosporin, a macrolide, a fluoroquinolone, a sulfonamide, a tetracycline, and an aminoglycoside, or a combination thereof. In an even further aspect, the antibiotic is selected from bacitracin, neomycin sulfate, polymixin B sulfate, penicillin, amoxicillin, cephalexin, erythromycin, clarithromycin, azithromycin, ciprofloxacin, levofloxacin, ofloxacin, co-trimoxazole, trimethoprim, tetracyclin, doxycycline, gentamicin, and tobramycin, or a combination thereof.

In a further aspect, the antibiotic consists essentially of bacitracin and polymixin B sulfate.

In a further aspect, the antibiotic consists essentially of bacitracin, polymixin B, and neomycin sulfate. In a still further aspect, the antibiotic consists essentially of bacitracin and neomycin sulfate. In yet a further aspect, the antibiotic consists essentially of polymixin B and neomycin sulfate.

In a further aspect, the antimicrobial is administered in an amount of from about 1 wt % to about 10 wt %. In a further aspect, the antimicrobial is administered in an amount of from about 1 wt % to about 8 wt %. In a still further aspect, the antimicrobial is administered in an amount of from about 1 wt % to about 6 wt %. In yet a further aspect, the antimicrobial is administered in an amount of from about 1 wt % to about 4 wt %. In an even further aspect, the antimicrobial is administered in an amount of from about 1 wt % to about 2 wt %. In a still further aspect, the antimicrobial is administered in an amount of from about 2 wt % to about 10 wt %. In yet a further aspect, the antimicrobial is administered in an amount of from about 4 wt % to about 10 wt %. In an even further aspect, the antimicrobial is administered in an amount of from about 6 wt % to about 10 wt %. In a still further aspect, the antimicrobial is administered in an amount of from about 8 wt % to about 10 wt %.

In various aspects, the first cannabinoid and the antimicrobial, together, are administered in a therapeutically effective amount (i.e. , the first cannabinoid and the antimicrobial are administered in a combinatorically therapeutically effective amount). In a further aspect, the first cannabinoid is administered in a therapeutically effective amount (i.e., the first cannabinoid is administered in an individually therapeutically effective amount).

In various aspects, the first cannabinoid and the antimicrobial are present as a composition. In a further aspect, the first cannabinoid and the antimicrobial are not present as a composition.

In various aspects, CBD and the antibiotic are present as a composition. In a further aspect, CBD and the antibiotic are not present as a composition.

In various aspects, CBG and the antibiotic are present as a composition. In a further aspect, CBG and the antibiotic are not present as a composition.

In various aspects, the first cannabinoid and the antimicrobial are administered sequentially. In a further aspect, the first cannabinoid and the antimicrobial are administered simultaneously.

In various aspects, CBD and the antibiotic are administered sequentially. In a further aspect, CBD and the antibiotic are administered simultaneously.

In various aspects, CBG and the antibiotic are administered sequentially. In a further aspect, CBG and the antibiotic are administered simultaneously.

In various aspects, the method further comprises topically administering to the wound an effective amount of a second cannabinoid, wherein the first cannabinoid and the second cannabinoid are different. In various further aspects, the composition further comprises an effective amount of a second cannabinoid, wherein the first cannabinoid and the second cannabinoid are different. In a further aspect, the second cannabinoid is selected from Δ9-tetrahydrocannabinol (THC), cannabinol (CBN), and cannabigerol (CBG), or a mixture thereof. In a still further aspect, the second cannabinoid is CBG. In a still further aspect, the second cannabinoid is CBG. In yet a further aspect, the second cannabinoid is THC. In an even further aspect, the second cannabinoid is CBN. In a still further aspect, the second cannabinoid is a mixture of two or more of THC, CBN, and CBG. In yet a further aspect, the second cannabinoid is a mixture of THC and CBN. In an even further aspect, the second cannabinoid is a mixture of THC and CBG. In a still further aspect, the second cannabinoid is a mixture of CBN and CBG. In yet a further aspect, the second cannabinoid is not a mixture of more than one cannabinoid.

In various aspects, the method further comprises topically administering to the epidermal wound an effective amount of a second cannabinoid selected from Δ9-tetrahydrocannabinol (THC), cannabinol (CBN), and cannabigerol (CBG), or a mixture thereof. In various further aspects, the composition further comprises an effective amount of a second cannabinoid selected from Δ9-tetrahydrocannabinol (THC), cannabinol (CBN), and cannabigerol (CBG), or a mixture thereof

In various aspects, the method further comprises topically administering to the epidermal wound an effective amount of a second cannabinoid selected from Δ9-tetrahydrocannabinol (THC), cannabinol (CBN), and cannabidiol (CBD), or a mixture thereof. In various further aspects, the composition further comprises an effective amount of a second cannabinoid selected from Δ9-tetrahydrocannabinol (THC), cannabinol (CBN), and cannabidiol (CBD), or a mixture thereof

In various aspects, the first cannabinoid and the second cannabinoid, together, are administered in a therapeutically effective amount (i.e. , the first cannabinoid and the second cannabinoid are administered in a combinatorically therapeutically effective amount). In a further aspect, the second cannabinoid is administered in a therapeutically effective amount (i.e., the second cannabinoid is administered in an individually therapeutically effective amount).

In various aspects, the first cannabinoid and the second cannabinoid are present as a composition. In a further aspect, the first cannabinoid and the second cannabinoid are not present as a composition.

In various aspects, the first cannabinoid and the second cannabinoid are administered sequentially. In a further aspect, the first cannabinoid and the second cannabinoid are administered simultaneously.

In various aspects, the first cannabinoid and/or the antimicrobial are present on an article, and wherein topically administering is via application of the article to the epidermal wound. In a further aspect, the first cannabinoid and/or the antimicrobial are present on a bandage or a wound dressing material, and wherein topically administering is via application of the bandage or the wound dressing material to the epidermal wound.

In various aspects, CBD and/or the antimicrobial are present on an article, and wherein topically administering is via application of the article to the epidermal wound. In a further aspect, CBD and/or the antibiotic are present on a bandage or a wound dressing material, and wherein topically administering is via application of the bandage or the wound dressing material to the epidermal wound.

In various aspects, CBG and/or the antimicrobial are present on an article, and wherein topically administering is via application of the article to the epidermal wound. In a further aspect, CBG and/or the antibiotic are present on a bandage or a wound dressing material, and wherein topically administering is via application of the bandage or the wound dressing material to the epidermal wound.

### 2. Use of Compositions

In one aspect, disclosed are uses of a disclosed composition. In a further aspect, a use relates to the manufacture of a medicament for the treatment of an epidermal wound in a subj ect.

Also provided are the uses of the disclosed compositions and products. In one aspect, disclosed are uses of at least one disclosed composition.

In a further aspect, the use relates to a process for preparing a pharmaceutical composition comprising a therapeutically effective amount of a disclosed composition or a product of a disclosed method of making, for use as a medicament.

In a further aspect, the use relates to a process for preparing a pharmaceutical composition comprising a therapeutically effective amount of a disclosed composition or a product of a disclosed method of making, wherein a pharmaceutically acceptable carrier is intimately mixed with a therapeutically effective amount of the composition.

In various aspects, the use relates to a treatment of an epidermal wound in a subject. In one aspect, the use is characterized in that the subject is a human.

In a further aspect, the use relates to the manufacture of a medicament for the treatment of an epidermal wound in a subject.

It is understood that the disclosed uses can be employed in connection with the disclosed compositions, methods, and kits. In a further aspect, the invention relates to the use of a disclosed composition in the manufacture of a medicament for the treatment of an epidermal wound in a mammal.

### 3. Manufacture of a Medicament

In one aspect, the invention relates to a method for the manufacture of a medicament for treating an epidermal wound in a subject having the epidermal wound, the method comprising combining a therapeutically effective amount of a first cannabinoid and an antimicrobial or a therapeutically effective amount of a disclosed composition with a pharmaceutically acceptable carrier or diluent.

As regards these applications, the present method includes the administration to an animal, particularly a mammal, and more particularly a human, of a therapeutically effective amount of the composition effective in the treatment of an epidermal wound. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The size of the dose also will be determined by the route, timing, and frequency of administration as well as the existence, nature, and extent of any adverse side effects that might accompany the administration of the composition and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed composition with a pharmaceutically acceptable carrier or diluent.

### E. KITS

In one aspect, disclosed are kits comprising a first cannabinoid and an antimicrobial and one or more of: (a) an agent known to treat an epidermal wound; (b) a bandage or a wound dressing material; (c) instructions for topically administering the first cannabinoid to an epidermal wound; and (d) instructions for treating an epidermal wound. Examples of skin ailments include, but are not limited to, burns, sores, lacerations, blisters, insect bites, surgical incisions, and ulcers.

In one aspect, disclosed are kits comprising a composition comprising CBD and an antimicrobial and one or more of: (a) an agent known to treat an epidermal wound; (b) a bandage or a wound dressing material; (c) instructions for topically administering the first cannabinoid to an epidermal wound; and (d) instructions for treating an epidermal wound.

In one aspect, disclosed are kits comprising a composition comprising CBG and an antimicrobial and one or more of: (a) an agent known to treat an epidermal wound; (b) a bandage or a wound dressing material; (c) instructions for topically administering the first cannabinoid to an epidermal wound; and (d) instructions for treating an epidermal wound.

Examples of agents known to treat skin ailments include, but are not limited to, emollients, keratolytics, local anesthetic agents, local antipruritic agents, antibacterial agents, antiviral agents, antifungal agents, anti-inflammatory agents, antiparasiticidal agents, debriding agents, antineoplastic agents, burn treatment agents, eczema agents, psoriasis agents, and agents known for the treatment of diabetic foot ulcers.

In a further aspect, the at least one compound and the at least one agent are co-formulated. In a further aspect, the at least one compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed vaporizable solution and/or thermally unstable agent and another component for delivery to a subject.

The previous description of the disclosed aspects is provided to enable any person skilled in the art to make or use the present disclosure. Various modifications to these aspects will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for treating an epidermal wound on a subject, the method comprising topically administering to the epidermal wound a therapeutically effective amount of a first cannabinoid and an antimicrobial.

2. The method of claim 1, wherein the epidermal wound is a bacterial infection.

3. The method of claim 1, wherein the epidermal wound is selected from a burn, a sore, a laceration, a blister, an insect bite, a surgical incision, and an ulcer.

4. The method of claim 1, wherein the first cannabinoid is selected from Δ9-tetrahydrocannabinol (THC), cannabidiol (CBD), cannabinol (CBN), and cannabigerol (CBG), or a mixture thereof.

5. The method of claim 1, wherein the first cannabinoid is CBD.

6. The method of claim 1, wherein the first cannabinoid is THC.

7. The method of claim 1, wherein the first cannabinoid is administered in an amount of from about 0.5 wt % to about 10 wt %.

8. The method of claim 1, wherein the antimicrobial is an antibiotic.

9. The method of claim 8, wherein the antibiotic is selected from a penicillin, cephalosporin, a macrolide, a fluoroquinolone, a sulfonamide, a tetracycline, and an aminoglycoside, or a combination thereof.

10. The method of claim 8, wherein the antibiotic consists essentially of bacitracin and polymixin B sulfate, or wherein the antibiotic consists essentially of bacitracin, polymixin B, and neomycin sulfate.

11. The method of claim 1, wherein the antimicrobial is an antifungal agent.

12. The method of claim 1, wherein the antimicrobial is administered in an amount of from about 1 wt % to about 10 wt %.

13. The method of claim 1, wherein the first cannabinoid and the antimicrobial are administered sequentially.

14. The method of claim 1, wherein the first cannabinoid and the antimicrobial are administered simultaneously.

15. The method of claim 1, wherein the first cannabinoid and/or the antimicrobial are present on a bandage or a wound dressing material, and wherein topically administering is via application of the bandage or the wound dressing material to the epidermal wound.

16. The method of claim 1, further comprising topically administering to the wound an effective amount of a second cannabinoid, wherein the first cannabinoid and the second cannabinoid are different.

17. A method for treating an epidermal wound on a subject, the method comprising topically administering to the epidermal wound a composition comprising CBD in an amount of from about 0.5 wt % to about 10 wt % and an antibiotic in an amount of from about 1 wt % to about 10 wt %, based upon the total weight of the composition administered, wherein the antibiotic consists essentially of bacitracin and polymixin B sulfate.

18. The method of claim 17, wherein CBD and/or the antibiotic are present on a bandage or a wound dressing material, and wherein topically administering is via application of the bandage or the wound dressing material to the epidermal wound.

19. A method for treating an epidermal wound on a subject, the method comprising topically administering to the epidermal wound a composition comprising CBG in an amount of from about 0.5 wt % to about 10 wt % and an antibiotic in an amount of from about 1 wt % to about 10 wt %, based upon the total weight of the composition administered, wherein the antibiotic consists essentially of bacitracin and polymixin B sulfate.

20. The method of claim 19, wherein CBG and/or the antibiotic are present on a bandage or a wound dressing material, and wherein topically administering is via application of the bandage or the wound dressing material to the epidermal wound.
